(19) United States
(12) Patent Application Publication
(10) Pub. No.: US 2026/0081020 A1
(43) Pub. Date: Mar. 19, 2026

(54) COMPUTER-IMPLEMENTED METHOD FOR CARRYING OUT A PALPATION EXAMINATION ON A PATIENT'S BODY PART AND SYSTEM WITH SENSORIAL GLOVE, COMPUTER PROGRAM AND ASSOCIATED STORAGE MEANS

(71) Applicant: GLOOMA LDA., BRAGA (PT)

(72) Inventors: Francisco Manuel FARIA DE VASCONCELOS NETO NOGUEIRA, ALCABIDECHE (PT); Frederico Maria PERALTA STOCK, ESTORIL (PT)

(21) Appl. No.: 19/107,991

(22) PCT Filed: Oct. 20, 2023
(86) PCT No.: PCT/PT2023/050038

§ 371 Date: Feb. 28, 2025

(30) Foreign Application Priority Data
Oct. 31, 2022 (PT) 118298

(51) Int. Cl. G16H 50/20; G16H 10/60; G16H 20/40; G16H 50/70

(52) U.S. Cl. CPC G16H 50/20; G16H 10/60; G16H 20/40; G16H 50/70

(57) ABSTRACT

The invention refers to a computer-implemented method and respective system (300), computer program and computer-readable storage means for carrying out a palpation examination on a patient's body part (1,2,3,4,5,D, E), wherein the examination is performed systematically and according to an examination tutorial which is presented on a first computer unit (50). A palpation glove (10) is utilized in the examination, which is based on a reference point (7) and has incorporated pressure sensors capable of measuring pressure data on the texture of the tissue to detect alterations (6) in this tissue. The data obtained by the sensors is communicated by a control unit (20) of the glove (10) for processing in a second computer unit (90) which determines whether or not there have been alterations (6) to the tissue under examination by comparing, for example, with data from prior examinations.

### TECHNICAL FIELD

The subject matter of the present application is comprised in computer-implemented inventions, namely a computer-implemented method for carrying out a palpation examination on a patient's body part and the system configured to run said method. More particularly, the present application refers to a method and a system that includes a sensorial glove for carrying out a palpation examination on a patient's body part, said body part being monitored for alterations in texture over time. The present application also refers to a computer program with logical instructions to run the method of the present application and to the respective computer-readable data storage means.

### BACKGROUND ART

Cancer is a disease that may appear through alterations in the texture of the patient's biological tissues, and can be treated with much higher success rates if detected at a precocious stage.

For example, breast cancer is the most common oncological disease among women, affecting over 2.1 million every year, and is also the most fatal among them. In Portugal, according to data from GLOBOCAN, 60 467 new diagnoses were recorded, and 30 168 deaths were attributable to breast cancer in 2020.

According to the World Health Organization (WHO), to improve the prognosis for women, the precocious detection of breast cancer is crucial, two strategies being recommended: precocious diagnosis and screening. One of the screening methods indicated is the breast self-examination, this method also being a form of precocious detection of any neoplasia that may appear in the breast tissue.

In a survey carried out among a population of 621 women aged over 25, 16.9% said they had never carried out self-palpation and 70.6% performed it less than once per month (the frequency recommended by the WHO). The main reasons given were: forgetfulness, not knowing how to do it correctly and insecurity in performing the self-examination. Many women do not feel confident to do the self-test and oftentimes do not know how to do it, nor do they do it systematically, continuously, periodically, so as to detect alterations in the texture of the breast in the most effective manner. Bearing in mind the answers obtained in the questionnaire, the inventors have developed a solution that will enable and facilitate the performance of the breast self-examination by the woman herself, but which can also be used by a carer or health professional, systematically, reliably and with a high degree of precision.

### PRIOR ART

North American Patent N^{o}. U.S. Pat. No. 9,9301,563 B2, by Hardy et al., USA, published on Apr. 5, 2016, discloses the pressure and depth sensing glove also on the thumb and on the index finger, wherein an alarm signal (audio, vibration, etc.) is emitted when the pressure on one of the sensors reaches a pre-programmed threshold, with a view to monitoring and or to training the user on the pressure exerted, for example, on the head of a newborn. The glove can be connected to a computer with an output for a user.

US patent application N^{o}. US 2018/0000348, by Bishara et al., USA, published on Jan. 4, 2018, discloses a glove with sensors and a method for determining the elasticity module of the skin or of a tissue of a patient.

On the other hand, patent application PT 118167, filed on Aug. 22, 2022 also by the same Applicant, which describes a method and system for examining the evolution of the texture of a biological tissue of a patient's organ by means of periodical palpation examinations of this biological tissue, by way of a palpation glove, wherein said glove has fingers and sensors on the fingers, does not fully solve the problem of a correct fastening of the sensors on the glove and, consequently, of the sensors under the user's fingers, such that the system identifies the precise location of any potential alteration in the texture of the patient's biological tissue, besides being limited to sensors aligned on the glove fingers, which constitutes the limited palpation coverage area.

Accordingly, the state of the art does not yet have a device and respective method for examining the evolution of the texture of a biological tissue of a patient's organ, for example, the breast, or the testicle, which makes it possible to monitor over time, in a systematic and reliable manner, the texture of this organ, precisely identifying the position/size and other characteristics of the alteration of this texture, if any, and enabling them to be identified at a precocious stage.

### SUMMARY OF INVENTION

The invention refers to a computer-implemented method for a carrying out a palpation examination of a patient's body part, and to a system that implements the method, wherein the system includes a palpation glove with piezoelectric pressure sensors and computational means to record and process the data obtained by the sensors. The palpation examination is preferably repeated systematically, periodically, consisting of a plurality of palpation examinations, carried out on the same patient's body part, in the same manner, in order to detect alterations in the texture thereof over time, alterations in size of any potential anomaly already existing and, consequently, precociously detect the appearance of a cancer so that the treatment can begin as soon as possible and the disease be treated, leading to higher cure rates. The plurality of examinations carried out on the same body part from the same patient produces values measured by a sensor or by a plurality of sensors placed on a sensorial glove by way of an examination that is preferably always carried out according to the same sequential method.

In a first aspect, the present invention refers to a computer-implemented method for a carrying out a palpation examination on a patient's body part, said body part being monitored for alterations in its texture over time, wherein the palpation examination utilizes a palpation glove (10), a first computer unit (50) with a palpation examination tutorial and a second computer unit.

In a second aspect, the present invention refers to a system configured to implement the method referred to above, wherein said system comprises a palpation glove equipped with a flexible and/or foldable board equipped with pressure sensors that obtain values, during a palpation examination, relating to the texture of the biological tissue under examination and/or to the size of any potential alteration of this texture or of an already existing anomaly, a first computer unit with a palpation examination tutorial and a second computer unit configured to run the steps of the method relating to the calculations of the values measured by the pressure sensors of the palpation glove.

In a third aspect, the present invention refers to a computer program product comprising a set of logical instructions capable of making the first computer unit and/or the second computer unit run steps of the method of the first aspect of the present invention.

In a fourth aspect, the present invention refers to a computer unit-readable storage means, comprising a set of logical instructions which, when run by a computer unit of the present invention, carry out steps of the method of the first aspect of the invention.

### SOLUTION TO PROBLEM

The present invention provides a solution that enables implementation of a palpation examination routine of a patient's body part, for example, the breast. By way of the palpation glove and the sensors that measure the texture of the tissue under the point pressed, following a tutorial that teaches the user to do the examination in the same way always, following the same palpation path, and by way of the computer units involved that perform the calculations relative to the values obtained by the sensors in order to find alterations over time, it is possible to precociously detect the appearance of benign or malignant nodules. In particular, the construction of this palpation glove enables the accurate and precise positioning of each sensor on the point examined, accurately and precisely enabling the identification of each point of the biological tissue over time in the various palpation examinations of the same body part of the same patient. Further, in order to enhance the precision with which the size of a potential nodule existing in the biological tissue under examination is determined, certain embodiments of the glove of the present invention have a plurality of sensors sufficiently adjacent to each other forming a mesh of sensors the enable the size of the existing nodule to be obtained with greater precision. Additionally, since the sensors are arranged on a sensor board, wherein the board may be flexible and even foldable, a larger contact area is possible with the body part under examination, and better adapted to the contours of the body. The present invention also includes, in some embodiments, the emission of warning messages to the user, through a computer unit, indicating the need to consult a doctor, for example, or to do more diagnosis examinations.

### ADVANTAGEOUS EFFECTS OF INVENTION

The method and system according to the present invention enable the patient, or a carer or health professional, to perform a palpation examination with a convenient frequency, wherein this periodical palpation examination enables the patient, or a carer or health professional, to precociously detect a nodule, or an alteration in the texture of the biological tissue under examination, which may lead to a significant reduction in the rate of cancers that are not detected precociously, for example, breast cancer.

Advantageously, in using the method and the system of the present invention, a user, for example, a woman, may carry out a self-palpation examination, in the comfort of her own home, wherein she is guided in the self-examination by an application on her mobile device, for example a cellphone, such that the examination is always carried out in the same way, which will enable the breast tissue to be monitored over time. Additionally, the system according to the invention includes a palpation glove which, by its construction, that is, with a finger zone common to the four fingers performing the examination, on which a flexible and/or foldable board with sensors is fastened, enables more precise positioning and also more extensive and molded to the contours of the body, of the sensors relative to each examination point on the body part, for example, the breast, which is under examination. In cases where there are already breast nodules, for example, also the method and system of the present invention enable the size of existing nodules to be monitored, such that, once again, it is possible to detect alterations in the texture thereof, in the exact position and in size.

Advantageously, the flexible board with sensors positioned inside the palpation glove according to the present invention enables the attachment of the sensors which are easy to assemble for mass production and, naturally, to guarantee that the electronic connections of the conductor wires to the sensors are well protected.

Since the sensor board is removable, each glove can be utilized with boards different, with different sensor arrangement geometries, so as to adapt to different parts of the patient's body or to different patients, further enabling the same glove with the same sensor board to be utilized by both hands of the user, since there are side openings in the glove to let the thumb through, as described in detail ahead.

### BRIEF DESCRIPTION OF DRAWINGS

With the purpose of providing an understanding of the principles according to the embodiments of the present invention, reference will be made to the embodiments illustrated in the figures and to the terminology used to describe them. In any case, it should be understood that there is no intention of limiting the scope of the present invention to the contents of the figures. Any subsequent alterations or modifications of the inventive characteristics illustrated herein, as well as any additional applications of the principles and embodiments of the invention illustrated, which would normally occur to a person skilled in the art having the knowledge of this specification, are considered as being within the scope of the claimed invention.

FIG. 1A illustrates a perspective view of an embodiment of the palpation glove according to the invention, with the glove placed on the hand of the user, before covering the user's fingers with the part of the glove containing the board with the sensors inside;

FIG. 1B illustrates another perspective view, of the same embodiment of the FIG. 1A, of the palpation glove according to the invention, with the glove placed on the hand of the user, with the user's fingers inserted into the part of the glove containing the board with the sensors inside;

FIG. 1C illustrates a perspective view of other embodiment of the palpation glove according to the invention, with the glove placed on the hand of the user, with the user's fingers inserted into the part of the glove containing the board with the sensors inside;

FIG. 2A illustrates an embodiment of the sensor board of the palpation glove according to the invention, in which the conductor wires connecting the sensors to the power and control unit of the glove are visible;

FIG. 2B illustrates other embodiments of the sensor board of the palpation glove according to the invention;

FIG. 3 illustrates a diagram of an embodiment of the power and control unit of the glove with its components;

FIG. 4 schematically illustrates a patient's body part undergoing the palpation examination, identifying the various palpation points that are part of the examination;

FIG. 5 illustrates a flowchart of an embodiment of the method according to the invention;

FIG. 6 schematically illustrates an embodiment of the system according to the invention and a patient's body part under palpation examination;

FIG. 7 schematically illustrates two possibilities of displaying results of a palpation examination on two interfaces of different users, after the calculation by a second computer unit.

### DESCRIPTION OF EMBODIMENTS

The technological solution of the present invention enables palpation examinations to be carried out on a patient's body part, on a routine basis and with precision, enabling alterations to be found in texture of the biological tissue examined over time. The present invention refers, in one aspect, to a system that includes a palpation glove equipped with a flexible and/or foldable board with pressure sensors at least that collaborate with the palpation examination by measuring pressure values representing the texture of the tissue under examination. By periodically applying a tutorial displayed on a user interface of a computer device according to the invention, the user can carry out the same examination, in a consistent manner, whereby enabling the accurate surveillance of one or more parts of the patient's body in terms of position and size of any alterations or nodules detected. Said tutorial teaches the user to follow the steps of the examination to be carried out, the examination will always be performed in the same way, so that the pressure exerted, or palpation, on the tissue under examination, is always at the same palpation point, when the user follows the tutorial. This tutorial comprises a sequence of instructions accompanied by images illustrating the positions of the glove at each palpation point. The tutorial instructions may include at least one instruction from the group comprised of:
positioning of the palpation glove in a first reference point of the body part under examination;
movement of the glove in a defined direction, for example, clockwise, up to the next point of examination;
palpation of the zone to be examined with the fingers pressing the pressure sensors;
continued execution of the examination path by the glove as far as the last point of examination on the patient's body part under examination;
wherein the tutorial instructions can be accompanied by images and/or signs illustrating the palpation path on the patient's body part that the user should follow.

The palpation glove according to the invention is made with one or more layers of an elastic mesh fabric, disinfectable at least on the outer layer(s). According to the invention, the palpation glove comprises at least one pressure sensor situated in the zone of the user's fingers of the glove, but preferably the palpation glove according to the invention comprises a plurality of sensors arranged on a sensor board, so that the sensors are, at least partially, in zones nearer the end of the user's fingers than the palm of the hand. The board with the sensors may be flexible and/or foldable and is inside a mesh bag which in turn is inside a mesh cover which envelops the user's fingers and, not being in direct contact with the user's fingers or exposed to the surface. The board with the sensors is removable enabling same glove to be used with sensor boards with different geometries in the arrangement of the sensors so as to enable adjustment to different parts of the body or to different users, pursuant to the size of the part under examination, its contours and other morphological characteristics of the body part under examination.

The glove according to the invention covers just the four fingers carrying out the palpation examination, being equipped with one or more openings for the thumb, wherein the board with the sensors can be symmetrical relative to the central vertical axis, and can operate with the user's left or right hand. The mesh fabric layers protect the sensors and the user's hand and are disinfectable. Advantageously, the glove can be used indiscriminately by either hand of the user with just one sensor or a set of sensors arranged on a sensor board.

The glove may have the sensors arranged such that, for example, one sensor is under each finger collaborating with the palpation examination or two sensors arranged under each finger of the user making a total of eight sensors, thus obtaining more reliable values in a palpation examination, by calculating the averages between additional individual values obtained by the piezoelectric sensors. The sensor board of the glove may also have over eight sensors, the board may have a plurality of sensors covering its entire surface so as to cover the largest area of palpation. In this case, when a sensor detects an anomaly, the data obtained by the adjacent sensors are used to confirm, on the one side, the existence of the anomaly and, on the other, its extent.

Said sensors are each connected to a wire which connects via USB, which may be of the C type, mini USB, micro USB, USB A type, among other models.

The size of said piezoelectric sensors may vary between 10 and 20 mm, but may be smaller. In certain embodiments, the sensor board may have just one larger sensor, for example 40 mm, or may have 20 mm sensors, for example, four sensors measuring 20 mm, aligned such that one sensor is under each one of the four fingers of the second to the fifth finger of the user's hand. These pressure sensors may be positioned in various ways and in different configurations, but there are preferably at least two sensors, as said, positioned under each of the second to fifth fingers of the hand.

The sensors may be positioned adjacently or 0 cm to a maximum of 2.5 cm apart from each other, never being overlaid. Preferably, the sensors are 0.1 cm apart.

Said piezoelectric pressure sensors have the function of retrieving pressure data and pressure differences from different textures of the human body, not requiring any other sensor to make this reading. However, the glove may also include other types of sensors such as, example, a gyroscope and/or an accelerometer which will help to provide the location of the sensors or of the glove relative to the position on the body part under examination. Said piezoelectric sensors may be of varied types, for example, they may comprise a brass or copper disk, and have piezoelectric ceramic (for example, lead zirconate titanate) at their center or may have monocrystal materials (for example, quartz crystals). Additionally, the sensors may also comprise a piezoelectric membrane made of PVDF (polyvinylidene fluoride) polymer with silver nanoparticle-printed electrodes.

The palpation glove according to the present invention comprises a casing containing a power and control unit. The casing of this power and control unit may be placed on the part lateral of the glove, next to the wrist, or in another zone of the glove, for example, on the back of the hand, inside the mesh-coated bag, and may be withdrawn from the mesh bag, for washing the glove.

The power and control unit is connected to the sensors by way of suitable ports, for example, of one of the USB types indicated above and may comprise a printed circuit board (PCB), a light source, for example, a LED RGB (Light Emitting Diode, Red Green Blue), a microprocessor, a communication module via Bluetooth, wi-fi or other suitable communication mode, for example, zigbee, which may be wired or wireless, and the energy source which may be a chargeable or non-rechargeable battery, the energy source being rechargeable by USB of the C type, mini USB, micro USB, USB A type, among others. In certain embodiments, this power and control unit may comprise a movement sensor which may be an accelerometer and/or gyroscope or other movement suitable sensor. This movement sensor may be integrated into said power and control unit coupled to the glove or may be in another location of the glove, preferably, the movement sensor is in the power and control unit.

Through the readings of the movement sensor, it is possible to calculate the angle in which the sensor is positioned, that is, the position of the glove relative to the body part which it is examining and accordingly, identify the path traversed by the pressure sensors on the patient's body part.

The movement sensor may be a 3-axis accelerometer that can measure static and dynamic acceleration forces. The gravitational force of the earth is an example of static force and the dynamic forces, which will be disregarded, can be caused by vibrations, small movements or shocks. The unit of measurement of acceleration is m/s². However, in this case, the readings of the accelerometer are expressed in G (gravity), and the value of the gravitational force of the earth is 1 G. Therefore, when the accelerometer is positioned on the xy plane, with the axis z pointing upwards, that is, in a direction opposite the gravitational force, the value measured by the sensor on the z axis z will be 1 G, and the values measured on the xy axes are OG because the gravitational forces are perpendicular to these axes and do not affect them. With these values, it is possible to calculate the angle wherein the sensor is positioned.

When the user is wearing the glove and is ready to begin the examination, he or she is requested by the tutorial to put the glove in a certain position, which shall be the starting block, and carry out a self-calibration action. The objective of this self-calibration, or reset, of the glove is precisely to guarantee that the values measured on the x, y and z axes are, on one of the 1 G axes and on the other two 0 axes, as they are perpendicular to the gravitational force. For example, in the case of a self-examination of the breast, if we request the user to place his or her wrist above the nipple perpendicularly, that is, the casing comprising the accelerometer, preferably, same comprising the power and control unit, it will be on the plane of the xz axes whereby guaranteeing that there is freedom for the hand to move horizontally, the value measured on the y axis will be 1 G, and on the other axes will be 0, as a person skilled in the art will understand. From here onwards, it is possible to know the value measured on each of the axes and to calculate the angles wherein the accelerometer is positioned, accordingly assisting to identify the path traversed by the palpation glove during the examination.

The palpation glove may also have a physical mark, for example, a circle of another color, which will help to position on the starting point of the palpation examination, a point that will serve as reference for the path to traverse in the examination, which will be important for interpreting the position of the pressure sensors when measuring any alteration to the tissue under examination.

The self-calibration or reset of the glove can be performed having as reference point a physical mark disposed on the glove, as described in the preceding paragraph, but may also be performed based on the other reference points, for example the index finger placed on the nipple while the power and control unit is steered towards a previously configured direction, for example with the downward directioning of the power and control unit, that is, positioning it in a frontal position relative to the ground. In other embodiments of the self-calibration or reset of the glove, the reference point can be any point located on the user's hand, if the accelerometer is arranged on the glove, whereby avoiding the need for a physical mark arranged on the glove.

The palpation glove according to the invention is connected, via the communication module Bluetooth, wi-fi or other suitable form of communication, wired or wireless, to a first computer unit on which the computer application is running, or set of logical instructions, according to the invention, wherein said first computer unit can be selected from the group comprised of a portable computer, a fixed computer, a tablet, a smartphone, a cellphone, a smartwatch.

When the palpation glove is turned on in the user interface, for example, a pressure button, communication is established between the power and control unit of the palpation glove and the first computer unit by means of wired or wireless communication means, wherein said power and control unit of the palpation glove is ready to send data relative to values measured by pressure sensors to the first computer unit, that is, it gets the sensors ready to be used. When pressed in the palpation examination, these sensors generate numerical values. The values may be different to 0 upon contacting the surface of the human body, and the movement of the sensors accompanies the movement of the hand where the glove is being used, continuing to generate values that may be different to 0.

By combining the values measured by the pressure sensors and by the movement sensor, when present, it is possible to determine the location on the patient's body under examination where any potential alteration in texture was detected. The embodiments which do not have a movement sensor in the palpation glove also enable this detection, but the correct positioning of the glove at each examination point is more dependent on the user correctly following the tutorial.

During the palpation examination, when an alteration is detected by the pressure sensors, that is, a value measured by one or more sensors is sent for processing in the second computer unit, since it is different to what was expected for that patient, for example, a baseline of patient values, the computer application, or set of logical instructions, it may be considered to have found the anomaly. For example, the baseline is a variation between 0 and 20, wherein the value 20 is considered a threshold based on which the values may represent the anomaly for a certain type, ethnicity, age, etc., of patient. In this case, if the value found, for example, is 30, the computer application according to the invention may consider this value the anomaly and suggest repeating the examination on the following day, in order to eliminate the hypothesis that the result is a statistically negligible value, considering a potential stress of the patient.

The values measured by the pressure sensors will vary from 0 to 1023, and detecting benign and malignant anomalies will depend on the baseline of each patient. It is known in the state of the art that a malignant anomaly is normally harder than a benign one, so it will exert more pressure on the sensors and will consequently result in higher values. For example, if the baseline of a woman is in the values around 30, if in the order of 70 are obtained, wherein 70 is a threshold l₁, this may mean a benign anomaly, but if the value is in the order of hundreds, that is, over a threshold l₂ greater than l₁, it may be a malignant anomaly. The values will depend on the type of anomaly and variables of each patient, such as, for example, age, the menstrual period and ethnicity, as well as other variables grounded on scientific and medical knowledge. Therefore, the baseline that will serve as reference for drawing conclusions on the palpation examination on the body part of a particular patient, will be specific to this patient and will depend on the biological characteristics of the patient, among other characteristics. The method and system according to the invention detect alterations based on this expected baseline for each patient and each body part under examination. This baseline can be obtained from a database known in the state of the art, with values for each type of patient, and/or can be a baseline built up over time with the data retrieved from the particular patient in question.

When the palpation examination results in suspicious values, screening questions can be asked such as “Are you in your menstrual period?”, “Have you changed the brand of your pill?” or “Have you started to take the pill?”, “Do you have symptoms that might indicate menopause?”, etc., so as to identify factors that might cause momentary alterations. If any of these factors exist, the user may be requested by the computer application at the end of the palpation examination to repeat the examination one or two weeks later, for example. If there are no potentially influencing factors, the user/patient is requested to repeat the examination in the zone where the nodule was detected and, in accordance with the type of anomaly, the user may receive a warning to consult with a doctor. In some embodiments of the present invention, this warning can be sent directly to a health service or a doctor, if the user is not the doctor.

In order to terminate the reading of values by the sensors, after the passage over the entire surface intended for examination and seeking alterations of the texture, that is, when the examination is concluded, the glove is disconnected, terminating the reading of values by the sensors.

When the user begins the palpation examination, following the instructions on the interface of the first computer unit, the data is retrieved by way of glove sensors and the data is transmitted via the USB type input as already referred to. Thereafter, this data is sent to the second computer unit via Bluetooth, wi-fi or other suitable means of communication, wired or wireless, for processing. The processing of the data includes a pre-processing which is followed by an analysis by the computer application or set of logical instructions according to the invention. Said pre-processing includes the removal of statistically irrelevant data and may comprise one or more steps selected from the group consisting of eliminating empty fields without sensor data, eliminating zeros that correspond to non-relevant data, correcting and/or eliminating any data considered invalid, the uniform formatting of the files that include data relating to readings and a correction of values related to malfunctioning sensors.

The step of uniform formatting of the files which include data relating to readings is executed, for example, by one or more procedures selected from the group comprising the standardization of the format or of the extension of the files, the standardization of the structure of the files regarding the listing and the sorting of data related to readings, for example with the formatting of the data so that they are structured in records or on a non-linear data structure tree. The step of uniform formatting of the files that include data relating to readings also can be executed by way of other procedures known to a person skilled in the art and commonplace in the state of the art.

The step of correcting values related to malfunctioning sensors can also be executed by way of procedures known to a person skilled in the art and commonplace in the state of the art, such as the alteration of the magnitude of the values outside the norm to have as basis a null and void value or substituting the values for a low variance normal Gaussian noise, if the sensor is irrecoverable.

The data is subsequently analyzed. This analysis includes the comparison between the data obtained in the examination concluded and a baseline, which may consist of data obtained from technical knowledge of the field or may consist of data obtained in prior examinations from the same patient and same body part. For example, if a first set of cdᵢ data refers to a baseline, which may be a set of data from a previous examination, for example, an examination made thirty days beforehand, of the same body part from the same patient, and a second set of cdᵢ₊₁ data refers to the data obtained in the examination now under analysis, for each sensor or sensor mapping Sⱼ, the set of logical instructions according to the invention calculates a difference between cdᵢ₊₁ and cdᵢ for each sensor or sensor mapping Sⱼ: [cdᵢ₊₁Sⱼ−cdᵢSⱼ]. Depending on the sensor geometry used on the flexible board, the calculation can be advantageously carried out for a mapping of the sensors with the texture measurement area of the healthy or anomalous tissue, wherein this mapping enables a comparison between sensor boards with different sensor arrangement geometries. This mapping and utilization of different sensor boards may enable greater flexibility in the palpation examination and also the confirmation of suspicious values, if the patient is unable to visit a doctor immediately. If that value is different to zero, it is compared with a pre-established threshold l₁. This threshold determines the possibility that the alteration found constitutes a tumor, for example. However, this tumor may be, for example, benign or may be malignant. The method and system according to the invention may manage to estimate whether the tumor found will likely be benign or malignant by comparing [cdᵢ₊₁Sⱼ−cdᵢSⱼ] with the threshold l₁, already mentioned, but also with a threshold l₂, different to l₁, wherein, if the value of [cdᵢ₊₁Sⱼ−cdᵢSⱼ] is above l₂, the alteration found may already signify a malignant tumor. Depending on this result, the set of logical instructions of the computer-implemented method according to the invention may give specific recommendations to the user, for example, for the patient to visit a doctor urgently or else for the patient to repeat the examination, so as to confirm the values found. Said thresholds l₁ and l₂ can be established for each set of patient/body part/examination.

If, on the other hand, the value of [cdᵢ₊₁Sⱼ−cdᵢSⱼ] found is below the threshold l₁, the result of the examination may return a message to the user with a content indicating that there were no alterations in the body part examined and recommending that the examination be repeated within a certain period, for example, in a month's time.

The analysis of the examination data also includes an average of the values of all the pressure sensors, after eliminating the invalid values made in the pre-processing of the data. The average obtained is then compared with an average data baseline for that patient's body part under examination in a similar way to the one presented above for each sensor or Sⱼ sensor mapping.

The data already processed and the corresponding result obtained from this processing can be displayed in the first computer unit, in the second computer unit or in another computer unit, for example, of a health system. In certain embodiments, the data may also not be displayed, being stored for subsequent consultation by whomever is authorized to consult same.

The present invention can be used by a human or animal health professional, for example, a veterinarian, or can be used by human or animal health carer.

In a preferred embodiment, the method and the system of the invention are used by a patient who is also the user, that is, the palpation examination is a self-palpation examination.

Before or after placing the palpation glove, the user may connect his or her smartphone, for example, or other computer apparatus, and may call a specific computer application according to the invention. This computer application may access data kept on another computer unit through wired or wireless communication means or may have the data kept in the same computer unit. By this specific computer application, the user may create a patient profile, introducing data such as species, age, weight, ethnicity, gender, date of most recent menstruation, existence of breast implants, medication currently taking, for example, contraceptives, reason for carrying out the examination, who the user is, if the same person as the patient or if another person, among others. Upon creating the patient profile, the user may also introduce data relative to the palpation examination to be carried out such as, for example, the examination date, the patient's body part under examination, the examination points (1,2,3,4,5) which make up the palpation examination or, in certain embodiments of the present invention, the examination points may automatically become available when the user identifies the body part to be examined, sequential order of examination, frequency of the examination, results of prior examinations, recipient for sending the results, among others.

Next, the user, with the glove on, turns on the power of the glove through a user interface, which may be, for example, a pressure button and checks whether the glove is connected. To indicate the possible status of the palpation glove, in certain embodiments of the present invention, the glove comprises, in its power and control unit, one or more light sources emitting light of different colors, for example, in the form of a LED RGB, wherein, in certain preferred embodiments, the different colors may signify the following:
1—glove disconnected, the LED is turned off;
2—glove is turned on, a dark-blue LED lights up, and stays lit up continuously (without blinking) until a new command;
3—the user presses the pressure button to begin the reading and self-calibration occurs, during which the LED lights up blinking green;
4—having completed self-calibration, the LED stays a green color continuously (without blinking) during the palpation examination;
5—when the examination termina, the LED returns to dark-blue continuously;
6—when the glove is disconnected, the LED switches off;
7—in the event of a weak battery to prevents a full examination from being performed, the LED light blinks red until the battery is put on charge;
8—during charging, the LED assumes a continuous yellow color;
9—once the battery is charged, the LED turns dark-blue.

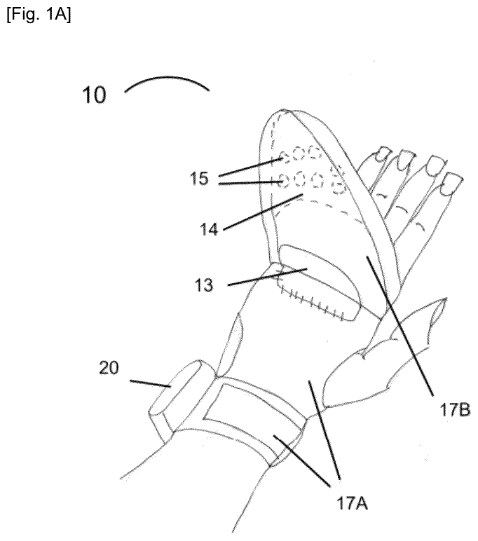

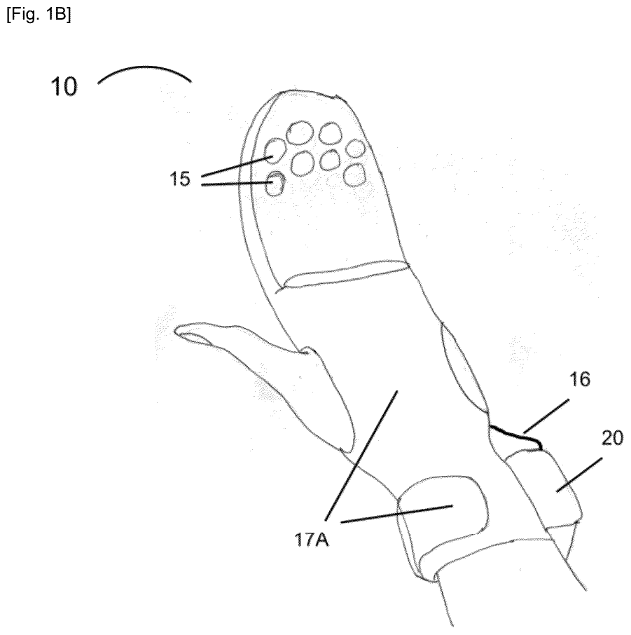

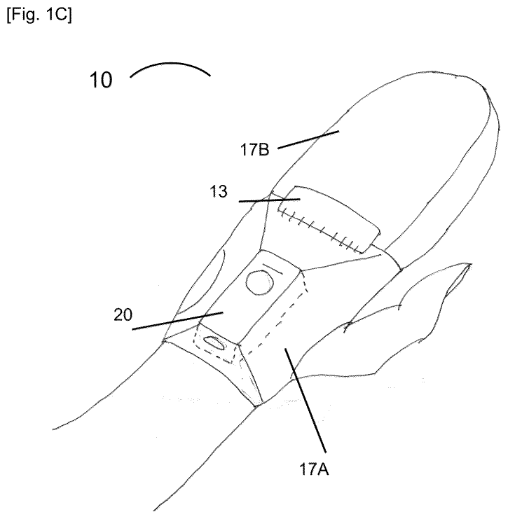

FIG. 1A shows a schematical representation of a perspective view of a palpation glove (10) according to preferred embodiments of the present invention. In the embodiment represented, eight sensors (15) are visible, placed on a sensor board (14) which is inside the finger cover (17B) of the palpation glove (10), wherein the sensors (15) are positioned aligned with the four fingers that remain inside the finger cover (17B) and are a suitable distance apart, between 0 cm and 2.5 cm, preferably a distance of 0.1 cm apart. Each sensor (15) may be between 10 mm and 20 mm, but may be smaller than 10 mm, and is connected to a connection wire (16) not visible in FIG. 1A, conductor, which has the function of communicating the reading of each pressure sensor to a power and control unit (20). This power and control unit (20) is inside a casing which in turn is inside a mesh bag and can be removed therefrom for the purpose of washing the glove (10). The glove (10) is comprised of two parts, a main glove body (17A) enveloping the hand and wrist of the user, and the main body (17A) can be equipped with a closing strip, such as, for example, a strip of velcro or another type, and the finger cover (17B), inside of which is a sensor board (14) with at least one sensor, but preferably a plurality of sensors (15) arranged therein, this finger cover (17B) being configured to “clothe” the user's fingers, positioning at the same time the sensors under the fingers so as to enable a palpation examination to be carried out by pressing the sensors (15) on the zone of the patient's body part under examination. The main body parts (17A) and finger cover (17B) of the palpation glove may be joined by sewing which may have a reinforcement hinge (13) that helps maintain the integrity of the fabric of which the glove is made. However, the main body parts (17A) and finger cover (17B) may also be joined by any other means known to person skilled in the art, enabling to the finger cover (17B) to be removable or not. In the main body part (17A) at the base of the fingers (not shown), there may be one or more traction handles (not shown) which allow the user to pull the glove and fit it on his or her hand so as to optimize the position of the sensors relative to his or her fingers to achieve the highest possible accuracy in the result of the palpation examination. Also next to the wrist of the glove, at the end of the main body (17B) proximal to the user, there may be one or more traction handles (not shown) to help fit the glove onto the hand. Additionally, the finger cover (17B) has sufficient length to be able to accommodate any fingernail length of the user of the glove, once again for improved adjustment of the glove and the sensors to the fingers with view to obtaining reliable and accurate results in the palpation examination. In FIG. 1A the glove is shown with the main body (17A) on the hand of the user and the finger cover (17B) not yet adjusted to the fingers. The finger cover (17B) may also have a joining element (not shown), for example, a piece of velcro, a button or a bracket, at its distal end, that is, on the part that covers the zone of the user's fingernails, for provisionally joining to the main body part (17A) so that the part (17B) does not swing when not placed on the user's fingers.

FIG. 1B shows a schematical representation of a perspective view of the inner side of the hand of the user of the palpation glove (10) of FIG. 1A, with the glove fully on and adjusted to the hand. In the position represented, the sensors (15) are not visible because they are inside the finger cover (17B), but are represented to facilitate the interpretation of their position under the user's fingers, prepared to carry out the palpation examination. This representation shows the set of connection wires (16) connecting the sensors (15) to the power and control unit (20) which may be joined which may be joined in a single cluster, as represented here.

FIG. 1C schematically represents another embodiment of the palpation glove (10) according to the present invention. In this embodiment of the invention, the power and control unit is placed on the back of the hand of the palpation glove and the glove is represented in the fully placed position, main body (17A) and finger cover (17B) in the position carrying out the palpation examination, on the hand of the user. Also in this embodiment, the glove may be equipped with a joining zone with hinge (13) to confer greater resistance in the movements of placing and withdrawing the finger cover (17B) and respective movements of adjusting the glove on the user's hand. In the embodiment represented in FIG. 1C, it is possible to see a pressure button (11) for interface with the user. This embodiment of the glove (10) may also be used on both the left and hand, since it has side openings for the thumb.

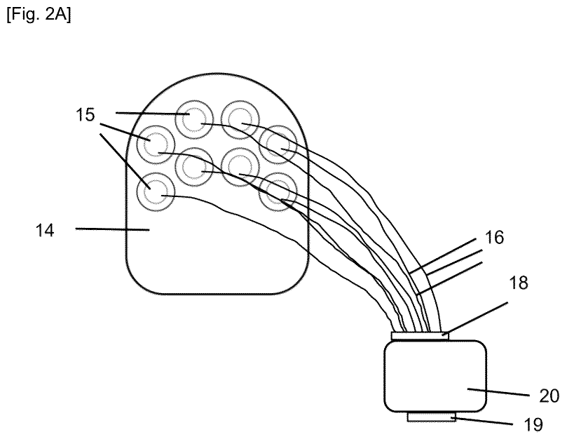

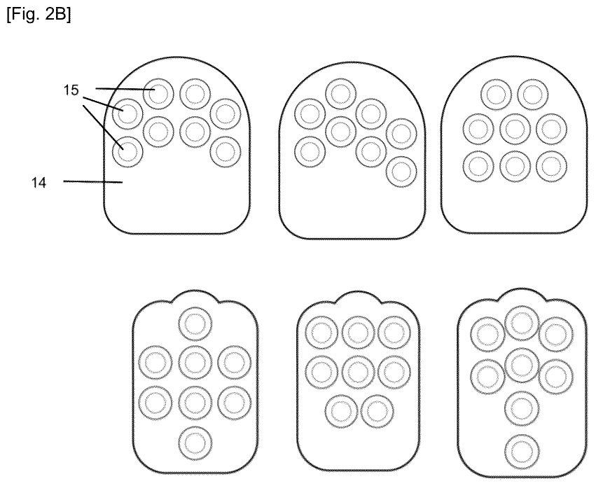

FIG. 2A illustrates an embodiment of the sensor board (14) in detail, for improved clarity. The sensor board is made of a flexible and/or foldable material, for example, an elastic fabric material. To each sensor (15) of the sensor board (14), a connection wire (16) is connected, which in turn is connected through a suitable port (18), for example, a USB port of one of the types indicated above, to the power and control unit (20). This power and control unit (20) also has an output (19) for the effects of charging a power supply, or battery, in embodiments wherein the battery is chargeable. The connection wires (16) are joined to the sensors by suitable means known to a person skilled in the art, such as, for example, by welding. An optional movement sensor (not represented) may also be included on the sensor board (14), which may be an accelerometer and/or a gyroscope.

FIG. 2B illustrates various embodiments of the board (14) with sensors (15) according to the invention. The arrangement of the sensors on the board can be symmetrical relative to a central vertical axis, such that they can be utilized with either hand of the user or may, in certain cases, not have a symmetrical arrangement of the sensors, wherein, in these cases, there may be two boards (14) for one same glove (10), wherein the boards are introduced and withdrawn according to the hand of the user who is performing the palpation examination.

The board of sensors is fastened inside the finger cover (17B) of the palpation glove (10) by suitable fastening means known by a person skilled in the art, such as, for example, by sewing, by positioning with velcro, by pressure buttons, among others.

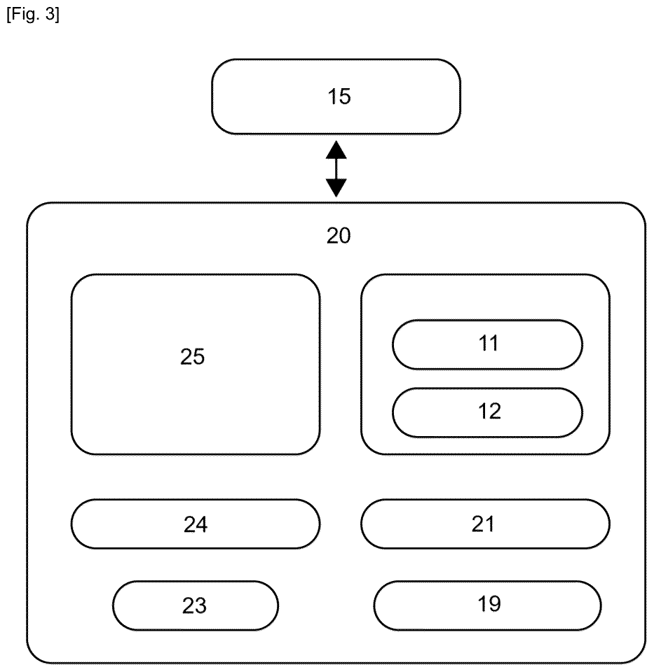

FIG. 3 illustrates a diagram of an embodiment of the power and control unit (20) of the glove (10). In the diagram of FIG. 2, it is possible to see the connection to the pressure sensors (15), the user interface (11), for example, a pressure button, which serves to connect and disconnect the glove, a light source (12), for example, a LED, to indicate the status of the palpation glove (10), a power supply (21), which may be a battery, chargeable or non-chargeable, which serves to power the electric circuits of the glove, which include the sensors (15) and the connection wires (16) and the respective input for charging (19) the power supply. Also represented in FIG. 3, in the unit (20), an optional movement sensor (23), which may be an accelerometer and/or a gyroscope, an exemplary communication unit (24), wired or wireless and a microcontroller (25) which serves for the analogical/digital conversion of the values measured by the sensors (15).

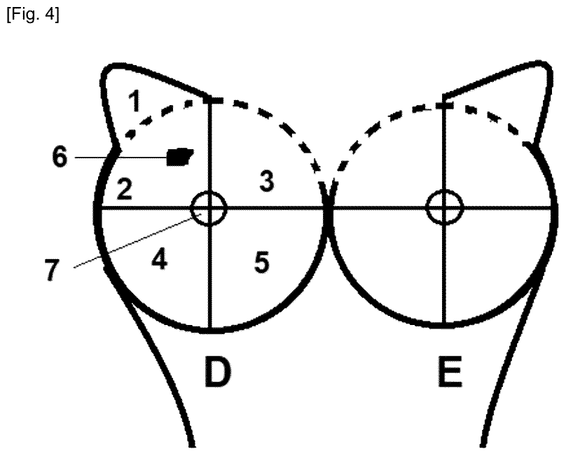

FIG. 4 schematically illustrates a patient's body part undergoing the palpation examination, which includes the left breast (E) and the right breast (D), identifying the various palpation points (1,2,3,4,5) that are part of the examination. The palpation points, in the body part example represented in FIG. 4, are the underarm tail (1), the upper outer quadrant (2), the upper inner quadrant (3), the lower outer quadrant (4) and the lower inner quadrant (5). FIG. 4 also shows a representation of the nipple (7), which may serve as reference point for the start of the examination and an alteration/anomaly (6) found by the sensors (15) in the upper outer quadrant (2) of the right breast during a palpation examination.

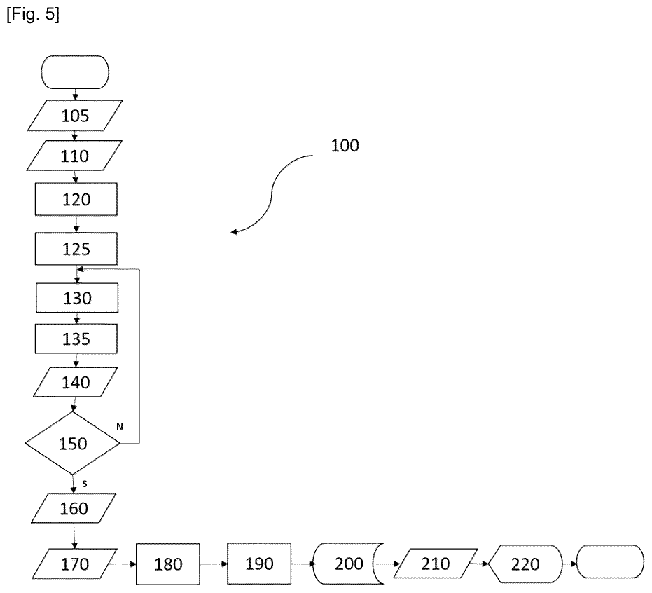

FIG. 5 illustrates a flowchart of an embodiment of the method according to the invention, wherein the method begins by the step of issuing a reminder (105) to the user that the data of the palpation examination is approaching, such that the examination is conducted according to a consistent and suitable frequency, for example, as advised by the WHO or by the doctor attending to the patient. The next step is the identification of the patient (110) through of a patient profile, with data of the patient and data of the examination to be carried out, wherein the patient data can be at least one of among species, age, weight, ethnicity, gender, date of most recent menstruation, existence of breast implants, medication currently taking, for example, contraceptives, and the patient profile may also include data on the examination to be carried out, such as, for example, examination date, patient's body part under examination, the examination points (1,2,3,4,5) that make up the palpation examination, sequential order of examination, frequency of the examination, results of prior examinations, recipient for sending the results. This identification (110) step also comprises the establishment of communication between the first computer unit (50) and the second computer unit (90) through communication means, for confirmation that identification is underway, of the patient and of the present palpation examination, and for retrieval of data stored in said database associated to the identification underway. The following step is the start of the palpation examination (120), wherein through user interfaces, both in the palpation glove and in the first computer unit, the user gives the start order, for example, connecting the glove and pressing a “start” zone on the screen of his or her smartphone, in the computer application displayed. At this point, the computer application presents the instructions relative to the examination, in a tutorial, wherein said instructions are as already defined above. The user, or the patient, in the case of a self-examination, first places the glove in the initial reference position indicated by the tutorial, confirming (125), in a “confirmation” zone on the screen of his or her smartphone, for example, that the initial position has been assumed and afterwards continues to follow the tutorial, making the palpation (130) according to these instructions and carrying out the palpation at each point indicated. Once each palpation point is concluded, through the input on the first computer unit interface, the user presses a zone de “confirmation” zone on the screen of his or her smartphone, for example, to confirm (135) the input of the data captured by the sensors relating to this palpation point. This confirmation also can be assumed by the computer application according to the invention, in certain embodiments, when it receives the data on the readings made by the sensors, after the dispatch of the following step. The power and control unit (20) of the glove proceeds to the dispatch (140) of the data to the first computer unit, which maintains it in memory for subsequent processing. When the last palpation point of the examination (150) arrives, the user confirms, on the interface (51), or observes a message appearing on said interface (51) that the examination is concluded (160) and the glove is disconnected on the user interface (11), for example on a pressure button. Next, the data of the examination concluded is sent (170) by the first computer unit to the second computer unit to be processed. The dispatch or communication means are means known by a person skilled in the art and have already been described in this application. Thereafter, the second computer unit executes the processing (180) of the data, which is comprised of two phases, a pre-processing and the analysis per se. The analysis of the data is based on a comparison of the data obtained in the examination concluded with baseline data, as already explained above in this application. Once processing is concluded, the results (190) are obtained of the palpation examination by the second computer unit, wherein the results may include information such as that mentioned below. Next is the storage step (200) of the results obtained from the palpation examination, wherein this storage can be performed in the second computer unit or in the first computer unit or else in another computer unit, for example, of a health system, for subsequent consultation. The results stored can also be sent (210) to the first computer unit or to a database of a health system or else to a computing device of a doctor or a caregiver, for example. Thereafter, the results can be displayed (220) on a user interface of the first computer unit or of other computer unit to which they were sent. The results of a palpation examination may include the information indicated ahead in this application.

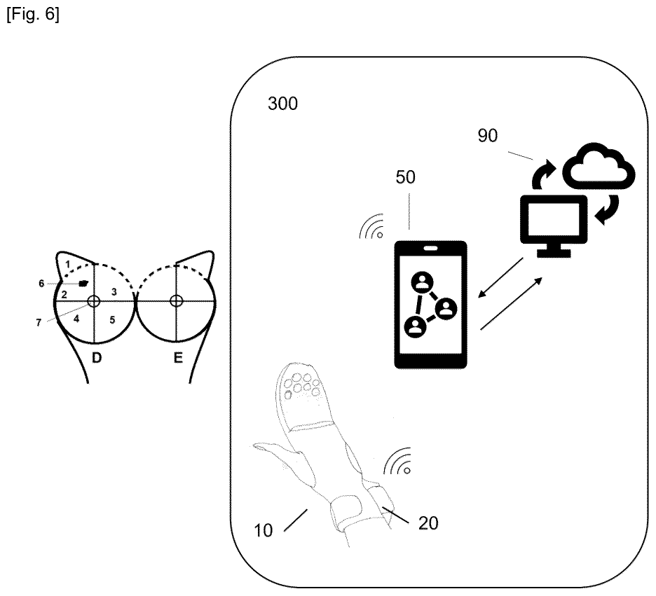

FIG. 6 schematically illustrates an embodiment of the system according to the invention, and also shows a patient's body part, the left breast (E) and the right breast (D) with an identifying of the examination points on the right. Specifically, the underarm tail (1), the upper outer quadrant of the breast (2), the upper inner quadrant of the breast (3), the lower outer quadrant of the breast (4) and the lower inner quadrant of the breast (5) are represented. Also represented is the nipple (7) which may serve as reference point for the start of the palpation examination and to guarantee that the examination is performed correctly, relative to the positioning at the palpation points. An alteration (6) of the texture of the breast tissue, which was found by the pressure sensors, is also represented in FIG. 6. In the representation of an embodiment of the system (300) according to the invention of FIG. 6, the first computer unit (50) is the smartphone of the user or of the patient receiving via wireless communication the values measured by the sensors at each examination point wherein the user, or the patient, confirms the data introduced. In turn, the smartphone communicates by suitable communication means, for example over the Internet, with the second computer unit (90), to send the data relating to a palpation examination already concluded, so that the second computer unit (90) proceeds to processing.

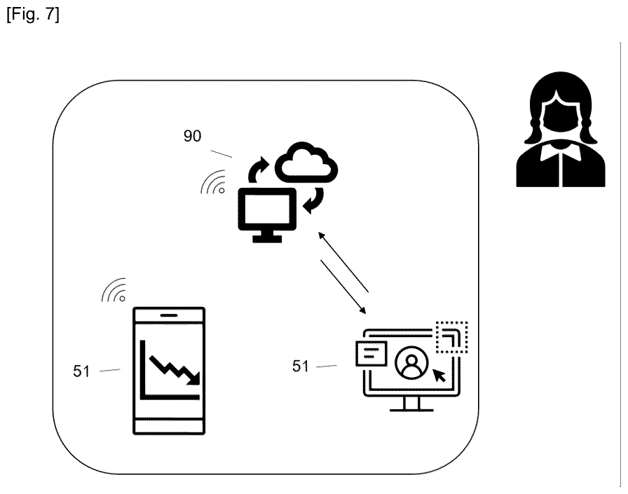

FIG. 7 schematically illustrates the part of the system (300) according to the invention and also the execution of steps of the method (100) according to the invention, illustrating the display, in non-exclusive form, of results of a palpation examination on two different and possible user interfaces (51), one on the smartphone of the patient and user and another on the computer screen of the doctor or of the health service to which the patient is associated, after receiving the results calculated by the second computer unit (90). The communication between the second computer unit (90) for dispatch of the results and the two different and possible user interfaces (51) can be made by means known in the art, wired or wireless.

In a first aspect, the invention refers to a computer-implemented method (100) for carrying out a palpation examination on a patient's body part, said body part being monitored for alterations in its texture over time, wherein the palpation examination utilizes a palpation glove (10), a first computer unit (50) comprising a palpation examination tutorial and a second computer unit (90) which runs a set of sequential and logical operations wherein said method (100) comprises the following steps:
a) identifying (110), in the first computer unit (50), the patient through a patient profile created in a database in a second computer unit (90), wherein the patient profile comprises patient data and data on the palpation examination to be carried out;
b) with the palpation glove (10) on and adjusted on a hand of the user, start (120) of the examination, carried out by the user, by means of a first user interface (51) of a first computer unit (50) and by means of a second user interface (11) of a palpation glove (10), wherein the first and second user interfaces of the first computer unit (50) and of the palpation glove (10), respectively, are at least one of the group comprised of pressure button, touch screen, microphone to pick up voice commands;
c) following the instructions from the examination tutorial displayed on the first user interface (51) of a first computer unit (50), the user confirms (125), by input in the first user interface (51), the correct position of the palpation glove on the first reference point (7) of the body part under examination;
d) movement of the glove to the following palpation point (1,2,3,4,5) indicated by the tutorial instructions and execution (130), by the user, of the palpation on the point indicated of the patient's body part;
e) palpation of the body part tissue under examination, through of the pressure exerted by the user's fingers on sensors (15) of the glove (10), exerting pressure with the fingers, so as to encompass the highest possible number of sensors arranged on a sensor board (14) inside the finger cover (17B) of the glove (10);
f) after palpation in the present palpation point, confirmation (135), by the user, or by a message displayed on the user interface (51), of the record of the values obtained by the sensors (15) arranged on the palpation glove (10), through the first user interface (51) of a first computer unit (50);
g) dispatch (140) in real time, by a power and control unit (20) of the palpation glove (10), of the values obtained by the sensors (15) in the present palpation point (1,2,3,4,5) to the first computer unit (50);
h) after confirmation (150) that the present palpation point (1,2,3,4,5) is the last palpation point (1,2,3,4,5), conclusion (160) of the palpation examination, by means of insertion, by the user, of a palpation examination end input in the first user interface (51), or by confirmation message displayed on the user interface (51) of a first computer unit (50) and by means of turning off, by the user, the examination glove (10) in the second user interface (11);
i) dispatch (170) of the data relative to the examination concluded, by the first computer unit (50), to the second computer unit (90), for processing (180);
j) processing (180) of the data relative to the examination concluded, by the second computer unit (90), wherein the processing includes the following substeps:
j1) pre-processing of the data obtained by the sensors in the examination concluded, wherein the pre-processing includes the removal of statistically irrelevant data;
j2) analysis of the data obtained in the terminated examination and comparison with data from a baseline associated to the patient, so as to detect alterations relating to this baseline; wherein the analysis of the data comprises the following substeps:
j21) making a table and/or a graph for a set of examination data and by sensor, wherein a first set of data cdᵢ refers to a baseline and a second set of data cdᵢ₊₁ refers to the data obtained in the examination under analysis, for each point Sⱼ, wherein
if [cdᵢ₊₁Sⱼ−cdᵢSᵢ]≠0 or above a threshold l₁ defined but below a threshold l₂ defined, the result of the examination indicates that an alteration was detected in the texture of the body part tissue under examination but that this alteration may be benign;
if [cdᵢ₊₁Sⱼ−cdᵢSⱼ] is above a threshold l₂ defined, wherein l₂>l₁, the result of the examination indicates that an alteration was detected in the texture of the body part tissue under examination and that this alteration may be malignant;
if [cdᵢ₊₁Sⱼ−cdᵢSⱼ]=0 or less than or equal to a threshold l₁ defined, the result of the examination indicates that no alteration was detected in the texture of the body part tissue under examination;
wherein the thresholds l₁ and l₂ are defined for each patient/examination pair;
j22) making an average of the valid data obtained by all the Sⱼ points, wherein the average obtained is compared with an average baseline of data for that patient's body part under examination;
wherein each Sⱼ point refers to the values of a sensor or of a mapping of sensors;
k) obtaining (190) the results of the palpation examination by the second computer unit (90); and
l) storage (200) of the results obtained in step k) in the second computer unit (90);
wherein steps d) to g) are repeated an n number of times for each palpation examination, wherein n is greater or equal to one, according to the instructions of the palpation examination tutorial.

In an embodiment of the first aspect, the invention refers to a computer-implemented method (100) as described above wherein the baseline associated to the patient is at least one selected from the group comprised of: baseline indicators of a patient-type similar to the patient under examination, obtained from databases or from prior technical knowledge and results of prior examination(s) of the same body part from the same patient.

In another embodiment of the first aspect, the invention refers to a computer-implemented method (100) as described above wherein the method further comprises the following steps:
m) dispatch (210) of the results obtained in step k) to the first computer unit (50), to a health system database, to a computer unit of a health professional or carer, wherein said dispatch of the results is carried out by wired or wireless communication means, for example, through a communications network such as the Internet; and
n) display (220) of the results obtained in step k) on a user interface (51) of the first computer unit (50) or on another user interface, for example, of a health professional, a carer or a health system.

In yet another embodiment of the first aspect, the invention refers to a computer-implemented method (100) as described above wherein the patient data comprises at least one of the following items of information: species, age, weight, ethnicity, gender, date of most recent menstruation, existence of breast implants, medication currently taking, for example, contraceptives; and the data on the palpation examination to be carried out comprises at least one of the following items of information: examination date, patient's body part under examination, the examination points (1,2,3,4,5) which make up the palpation examination, sequential order of examination, frequency of the examination, results of prior examinations, recipient for sending the results.

In another embodiment of the first aspect, the invention refers to a computer-implemented method (100) as described above wherein the results from step k) of obtaining the results include at least one item of information from the group comprised of:
identifying the presence of a possible undesirable alteration (6) in the texture of the tissue;
identifying the presence of a possible desirable alteration in the texture of the tissue;
identifying the presence/absence of alterations in the body part examined relative to a baseline;
characterization of the size/form/position or of another characteristic of the alteration found (6) on the patient's body part;
recommendation for immediate repetition of the examination;
recommendation for repetition of the examination within a set timeframe, for example, within one week, two weeks, within one month or three months;
recommendation to contact a health professional immediately or not immediately, for example, a doctor;
recommendation to carry out complementary diagnosis examination(s);
display of the automatically scheduled appointment date in the patient's health system with a message requesting confirmation; and
the information that the anomaly detected in the examination prior to this one persists, the user questioning whether the patient has already consulted the doctor.

In yet another embodiment of the first aspect, the invention refers to a computer-implemented method (100) as described above wherein the method (100) further comprises the following step, prior to step a):
a0) issuance (105) of a message-reminder to the user advising that the next examination should be carried out on a certain date in the near future, for example, in three days'time, in a day.

In another embodiment of the first aspect, the invention refers to a computer-implemented method (100) as described above wherein the patient is a mammal belonging to the group comprised of primates, horses, cattle, sheep, goats, canines, felines, cetaceans, pigs, deer, preferably the patient is a human primate.

In yet another embodiment of the first aspect, the invention refers to a computer-implemented method (100) as described above wherein the patient's body part in palpation examination pertains to the group comprised of breast, testicle, prostate, abdomen or other body part liable to be palpated, preferably the patient's body part is the breast. Palpation of the abdomen may contribute to detecting potential benign and malignant anomalies in the kidneys or spleen.

In another embodiment of the first aspect, the invention refers to a computer-implemented method (100) as described above wherein the user is the very patient, a human health carer, a human health professional, an animal health carer or an animal health professional, wherein preferably the user is the very patient or a human health professional and even more preferably the user is the very patient, that is, the palpation examination is a self-palpation examination.

In a second aspect, the invention refers to a system (300) configured to implement the method (100) as defined previously wherein the system (300) comprises:
a palpation glove (10) to be used by a user in a palpation examination of a patient's body part, wherein the palpation glove (10) comprises:
a main body (17A) of a palpation glove (10) wherein the main body is made of an elastic fabric mesh, the main body (17A) configured to envelop the part of the hand of the user excluding the fingers and to envelop at least partially the user's wrist;
the finger cover (17B) of a palpation glove (10), wherein the finger cover (17B) is made of an elastic fabric mesh and is joined to the main body (17A) by a joining zone and hinge (13), wherein the finger cover (17B) accommodates in a fixed position, on its inside, a sensor board (14);
a sensor board (14) comprising one or more pressure sensors (15) arranged to be under the user's fingers during the palpation examination and wherein the sensor board (14) is flexible and/or foldable;
one or more pressure sensors (15), preferably eight or more pressure sensors (15), wherein at least two pressure sensors (15) are under the user's fingers which are enveloped by the finger cover (17B) of the glove (10) during the palpation examination, wherein the largest pressure sensors (15) are up to 40 mm, preferably up to 20 mm and more preferably up to 15 mm;
power and control unit (20) which is coupled to the palpation glove (10), wherein the power and control unit (20) comprises:
a second user interface (11), for example, a pressure button, a tactile zone, an audio commands detection microphone;
at least one light source (12) indicative of a status of the palpation glove (10);
an input (19) for charging an electric power source (21);
an electric power source (21);
a communication unit (24), wired or wireless, with a first computer unit (50);
a microcontroller (25) to make the conversion between the analogical signals that the sensors (15) record into digital signals;
connection wires (16); wherein each connection wire (16) is connected to a sensor (15) at one end, and at the other end is connected to the power and control unit (20) through a suitable input (18);
first computer unit (50), wherein the first computer unit (50) comprises:
a first user interface (51) configured to display information to the user, for example, configured to display instructions on a palpation examination tutorial and receive input from the user, wherein the first computer unit (50) is selected from the group comprised of: portable computer, fixed computer, tablet, smartphone, smartwatch;
wherein the first computer unit (50) is configured to communicate with the power and control unit (20) of the palpation glove (10) and a second computer unit (90), by wired or wireless communication means;
second computer unit (90), wherein the second computer unit (90) is a device selected from the group comprised of: server, local network computer, remote cloud computer, portable computer, fixed computer, tablet, smartphone or any other computing device accessible through a communications network such as, for example the Internet, wherein the second computer unit (90) is configured to communicate with the first computer unit (50) by wired or wireless communication means.

In an embodiment of the second aspect, the invention refers to a system (300) as described above wherein the second computer unit (90) is integrated in the power and control unit (20) of the palpation glove (10) or is integrated in the first computer unit (50).

In another embodiment of the second aspect, the invention refers to a system (300) as described above wherein the pressure sensors (15) of the palpation glove (10) are arranged so as to maintain between themselves a distance d, wherein d pertains to the interval 0 cm to 2.5 cm and preferably d is 0.1 cm.

In another embodiment of the second aspect, the invention refers to a system (300) as described above wherein the palpation glove (10) comprises, in its main body (17A), two side openings for the thumb, so as to enable the user to utilize the palpation glove (10) with the left hand or with the right hand, and wherein the sensors (15) have an arrangement on the board (14) which is symmetrical relative to a central vertical axis of the flexible board (14) of the palpation glove (10). In yet another embodiment of the second aspect, the invention refers to a system (300) as described above wherein the palpation glove (10) further comprises a movement sensor (23), wherein the movement sensor (23) is placed on the board (14) or is placed on the power and control unit (20) and wherein the movement sensor (23) is selected from at least one of the group comprised of an accelerometer or a gyroscope.

In another embodiment of the second aspect, the invention refers to a system (300) as described above wherein the palpation glove (10) further comprises a mark configured to assist the user to position the glove correctly on the reference point (7) of the patient's body part at the start of each palpation examination.

In another embodiment of the second aspect, the invention refers to a system (300) as described above wherein the light source (12) is configured to emit, permanently or intermittently, one or more indicative colors, wherein the colors of the light source indicate at least one of the following statuses of the palpation glove (10):
turned off status;
turned on status awaiting new command;
status ready to start palpation examination;
self-calibration;
palpation examination concluded;
power supply (21) with low charge;
power supply (21) charging;
power supply (21) charged, palpation glove (10) ready to operate.

In a third aspect, the invention refers to a computer program product comprising a set of logical instructions which, when the program is run by a computer, for example by the first computer unit (50) or by the second computer unit (90) of the system as defined in any one of claims 10 to 16, the computer performs the steps j) to l) of the method as defined in any one of claims 1 to 9.

In a fourth aspect, the invention refers to a computer-readable storage means comprising a set of logical instructions which, when run by a computer, for example by the first computer unit (50) or by the second computer unit (90) of the system as defined in any one of claims 10 to 16, the computer performs the steps j) to l) of the method as defined in any one of claims 1 to 9.

### DEFINITIONS

As used in this application, the term “patient” refers to the person or animal whose body part is to undergo the palpation examination, while the term “user” refers to the person that is to put on the glove and to carry out the palpation examination. The “patient” and the “user” may coincide to be the same person, which will be a case of self-palpation examination.

As used in this application, the term baseline refers to a set of values-reference associated to a certain patient and respective body part under palpation examination, wherein this baseline of values will serve as reference for the computer application, or set of logical instructions, according to the present invention, detect alterations in the texture of the body part under examination, and this baseline can be obtained from databases, known in the state of the art or else from learning over time from data retrieved to the patient and respective body part in question.

As used throughout this patent application, the expression “or” is used in the inclusive sense, unless the exclusive sense is clearly defined in a specific situation. In this context, a sentence of the type “X uses A or B” must be interpreted as including all the pertinent inclusive combinations, for example “X uses A”, “X uses B”and “X uses A and B”.

As used throughout this patent application, the indefinite article “a”, “an” or “one” must be interpreted generally as “one or more”, unless the sense of a singular embodiment is clearly defined in a specific situation.

As presented in this specification, the expressions listed with examples must be interpreted with the purpose of illustrating an example and not indicating a preference.

The subject matter described above is provided as an illustration of the present invention and must not be interpreted so as to limit it. The terminology used with the purpose of describing specific embodiments, according to the present invention, must not be interpreted to limit the invention. As used in the specification, the definite and indefinite articles, in their singular form, are intended to be interpreted as also including the plural forms, unless the context of the description explicitly indicates otherwise. It will be understood that the expressions “comprise” and “include”, when used in this specification, specify the presence of the characteristics, the elements, the components, the steps, and the related operations, however, they do not exclude the possibility of other characteristics, elements, components, steps, and operations also being contemplated.

All the alterations, providing that they do not modify the essential characteristics of the claims that follow, must be considered as being within the scope of protection of the present invention.

### LIST OF REFERENCE INDICATIONS

1—underarm tail of a patient under examination
2—upper outer quadrant of the breast
3—upper inner quadrant of the breast
4—lower outer quadrant of the breast
5—lower inner quadrant of the breast
6—nodule detected by the sensors
7—nipple
10—palpation glove
11—user interface of the palpation glove
12—light source for indicating status of the glove
13—joining zone with hinge made of elastic material enabling the opening of the glove with improved handling
14—flexible and/or foldable board with sensors
15—pressure sensors
16—connecting conductor wires
17A—main body of the elastic mesh glove for retainment and adjustment with openings for the thumb
17B—elastic mesh zone that covers the fingers and contains the board with the sensors on the inside
18—data transmission input
19—battery charge input
20—power and control unit
21—battery (electric power source)
23—movement sensor (optional)
24—wireless communication module (Bluetooth)
25—microcontroller
50—first computer unit
51—user interface of the first computer unit
90—second computer unit
100—method according to the invention
105—emission of a reminder to the user that the examination date is approaching
110—identifying the patient through a patient profile, with data of the patient and data of the examination to be carried out
120—start of the palpation examination, through user interfaces both in the palpation glove and in the first computer unit
125—confirmation of the correct position of the palpation glove on the first reference point of the body part under examination
130—following the tutorial instructions, carry out the palpation examination at the palpation point indicated
135—confirmation, through the input on the first computer unit interface, of the values measured by the sensors
140—dispatch of the glove data to the first computer unit
150—is this the last palpation point of the examination?
160—conclusion of the examination introducing end of examination instruction on the user interfaces both from the first computer unit and in the palpation glove
170—dispatch of the data obtained in the examination concluded to the second computer unit, by the first computer unit
180—processing of the data by the second computer unit including the pre-processing of the data and the analysis of the data
190—obtaining the results of the palpation examination by the second computer unit
200—storage of the results obtained of the palpation examination
210—dispatch of the results obtained to the first computer unit or to a health system database
220—displaying the results of the examination on a user interface
300—system according to the invention
D—right breast
E—left breast

### LIST OF CITATIONS

List of citations follows:

### Patent Literature

PTL1: North American Patent N^{o}. US 9,9301,563 B2, by Hardy et al., USA, published on Apr. 5, 2016.
PTL2: North American Patent Application N^{o}. US 2018/0000348, by Bishara et al., USA, published on Jan. 4, 2018.
PTL3: Portuguese Patent Application PT 118167 filed on Aug. 22, 2022, by the Applicant.

### Non Patent Literature

NPL1: Sung H, Ferlay J, Siegel R L, et al. Global cancer statistics 2020: GLOBOCAN estimates of incidence and mortality worldwide for 36 cancers in 185 countries. CA Cancer J Clin. 2021; 71:209249 accessed on May 9, 2022.

1. A computer-implemented method (100) for carrying out a palpation examination on a patient's body part, said body part being monitored for alterations in its texture over time, wherein the palpation examination utilizes a palpation glove (10), a first computer unit (50) comprising a palpation examination tutorial and a second computer unit (90) which runs a set of sequential and logical operations wherein said method (100) is characterized by comprising the following steps:
a) identifying (110), in the first computer unit (50), the patient through a patient profile created in a database in a second computer unit (90), wherein the patient profile comprises patient data and data on the palpation examination to be carried out;
b) with the palpation glove (10) on and adjusted on a hand of the user, start (120) of the examination, carried out by the user, by means of a first user interface (51) of a first computer unit (50) and by means of a second user interface (11) of a palpation glove (10), wherein the first and second user interfaces of the first computer unit (50) and of the palpation glove (10), respectively, are at least one of the group comprised of pressure button, touch screen, microphone to pick up voice commands;
c) following the instructions from the examination tutorial displayed on the first user interface (51) of a first computer unit (50), the user confirms (125), by input in the first user interface (51), the correct position of the palpation glove on the first reference point (7) of the body part under examination;
d) movement of the glove to the following palpation point (1,2,3,4,5) indicated by the tutorial instructions and execution (130), by the user, of the palpation on the point indicated of the patient's body part;
e) palpation of the body part tissue under examination, through the pressure exerted by the user's fingers on sensors (15) of the glove (10), exerting pressure with the fingers, so as to encompass the highest possible number of sensors arranged on a sensor board (14) inside the finger cover (17B) of the glove (10);
f) after palpation in the present palpation point, confirmation (135), by the user, or by a message displayed on the user interface (51), of the record of the values obtained by the sensors (15) arranged on the palpation glove (10), through the first user interface (51) of a first computer unit (50);
g) dispatch (140) in real time, by a power and control unit (20) of the palpation glove (10), of the values obtained by the sensors (15) in the present palpation point (1,2,3,4,5) to the first computer unit (50);
h) after confirmation (150) that the present palpation point (1,2,3,4,5) is the last palpation point (1,2,3,4,5), conclusion (160) of the palpation examination, by means of insertion, by the user, of a palpation examination end input in the first user interface (51), or by confirmation message displayed on the user interface

(51) of a first computer unit (50) and by means of turning off, by the user, the examination glove (10) in the second user interface (11);
i) dispatch (170) of the data relative to the examination concluded, by the first computer unit (50), to the second computer unit (90), for processing (180);
j) processing (180) of the data relative to the examination concluded, by the second computer unit (90), wherein the processing includes the following substeps:
j1) pre-processing of the data obtained by the sensors in the examination concluded, wherein the pre-processing includes the removal of statistically irrelevant data;
j2) analysis of the data obtained in the terminated examination and comparison with data from a baseline associated to the patient, so as to detect alterations relating to this baseline; wherein the analysis of the data comprises the following substeps:
j21) making a table and/or a graph for a set of examination data and by sensor, wherein a first set of data cdᵢ refers to a baseline and a second set of data cdᵢ₊₁ refers to the data obtained in the examination under analysis, for each point Sⱼ, wherein
if [cdᵢ₊₁Sⱼ₋cdᵢSⱼ]≠0 or above a threshold l₁ defined but below a threshold l₂ defined, the result of the examination indicates that an alteration was detected in the texture of the body part tissue under examination but that this alteration may be benign;
if [cdᵢ₊₁Sⱼ₋cdᵢSⱼ] is above a threshold l₂ defined, wherein l₂>l₁, the result of the examination indicates that an alteration was detected in the texture of the body part tissue under examination and that this alteration may be malignant;
if [cdᵢ₊₁Sⱼ₋cdᵢSⱼ]=0 or less or equal to a threshold l₁ defined, the result of the examination indicates that no alteration was detected in the texture of the body part tissue under examination;
wherein the thresholds l₁ and l₂ are defined for each patient/examination pair;
j22) making an average of the valid data obtained by all the Sⱼ points, wherein the average obtained is compared with an average baseline of data for that patient's body part under examination;
wherein each Sⱼ point refers to the values of a sensor or of a mapping of sensors;
k) obtaining (190) the results of the palpation examination by the second computer unit (90); and
l) storage (200) of the results obtained in step k) in the second computer unit (90);
wherein steps d) to g) are repeated an n number of times for each palpation examination, wherein n is greater or equal to one, according to the instructions of the palpation examination tutorial.

2. The computer-implemented method (100) according to claim 1, characterized in that the baseline associated to the patient is at least one selected from the group comprised of: baseline indicators of a patient-type similar to the patient under examination, obtained from databases or from prior technical knowledge and results of prior examination(s) of the same body part from the same patient.

3. The computer-implemented method (100) according to claim 1 or with claim 2, characterized by further comprising the following steps:
k) dispatch (210) of the results obtained in step k) to the first computer unit (50), to a health system database, to a computer unit of a health professional or carer, wherein said dispatch of the results is carried out by wired or wireless communication means, for example, through a communications network such as the Internet; and
l) display (220) of the results obtained in step k) on a user interface (51) of the first computer unit (50) or on another user interface, for example, of a health professional, a carer or a health system.

4. The computer-implemented method (100) according to any one of preceding claim 1, characterized in that the patient data comprises at least one of the following items of information: species, age, weight, ethnicity, gender, date of most recent menstruation, existence of breast implants, medication currently taking, for example, contraceptives; and in that the data on the palpation examination to be carried out comprises at least one of the following items of information: examination date, patient's body part under examination, the examination points (1,2,3,4,5) that make up the palpation examination, sequential order of examination, frequency of the examination, results of prior examinations, recipient for sending the results.

5. The computer-implemented method (100) according to claim 1, characterized in that the results of step k) of obtaining the results include at least one item of information from the group comprised of:
identifying the presence of a possible undesirable alteration (6) in the texture of the tissue;
identifying the presence of a possible desirable alteration in the texture of the tissue;
identifying the presence/absence of alterations in the body part examined relative to a baseline;
characterization of the size/form/position or of another characteristic of the alteration found (6) on the patient's body part;
recommendation for immediate repetition of the examination;
recommendation for repetition of the examination within a set timeframe, for example, within one week, two weeks, within one month or three months;
recommendation to contact a health professional immediately or not immediately, for example, a doctor;
recommendation to carry out complementary diagnosis examination(s);
display of the automatically scheduled appointment date in the patient's health system with a message requesting confirmation; and
the information that the anomaly detected in the examination prior to this one persists, the user questioning whether the patient has already consulted the doctor.

6. The computer-implemented method (100) according to claim 1, characterized by further comprising the following step, prior to the step a):
a0) issuance (105) of a message-reminder to the user advising that the next examination should be carried out on a certain date in the near future, for example, in three days'time, in a day.

7. The computer-implemented method (100) according to claim 1, characterized in that the patient is a mammal belonging to the group comprised of primates, horses, cattle, sheep, goats, canines, felines, cetaceans, pigs, deer, preferably the patient is a human primate.

8. The computer-implemented method (100) according to claim 1, characterized in that the patient's body part under palpation examination pertains to the group comprised of breast, testicle, prostate, abdomen or other body part liable to be palpated, preferably a patient's body part is the breast.

9. The computer-implemented method (100) according to claim 1, characterized by the user is the very patient, a human health carer, a human health professional, an animal health carer or an animal health professional, wherein preferably the user is the very patient or a human health professional and even more preferably the user is the very patient, that is, the palpation examination is a self-palpation examination.

10. A system (300) configured to implement the method as defined in claim 1, characterized by comprising:
a palpation glove (10) to be used by a user in a palpation examination of a patient's body part, wherein the palpation glove (10) comprises:
a main body (17A) of a palpation glove (10) wherein the main body is made of an elastic fabric mesh, the main body (17A) configured to envelop the part of the hand of the user excluding the fingers and to envelop at least partially the user's wrist;
the finger cover (17B) of a palpation glove (10), wherein the finger cover (17B) is made of an elastic fabric mesh and is joined to the main body (17A) by a joining zone and hinge (13), wherein the finger cover (17B) accommodates in a fixed position, on its inside, a sensor board (14);
a sensor board (14) comprising one or more pressure sensors (15) arranged to be under the user's fingers during the palpation examination and wherein the sensor board (14) is flexible and/or foldable;
one or more pressure sensors (15), preferably eight or more pressure sensors (15), wherein at least two pressure sensors (15) are under the user's fingers which are enveloped by the finger cover (17B) of the glove (10) during the palpation examination, wherein the largest pressure sensors (15) are up to 40 mm, preferably up to 20 mm and more preferably up to 15 mm;
power and control unit (20) which is coupled to the palpation glove (10), wherein the power and control unit (20) comprises:
a second user interface (11), for example, a pressure button, a tactile zone, an audio commands detection microphone;
at least one light source (12) indicative of a status of the palpation glove (10);
an input (19) for charging an electric power source (21);
an electric power source (21);
a communication unit (24), wired or wireless, with a first computer unit (50);
a microcontroller (25) to make the conversion between the analogical signals that the sensors (15) record into digital signals;
connection wires (16); wherein each connection wire (16) is connected to a sensor (15) at one end, and at the other end is connected to the power and control unit (20) through a suitable input (18);
first computer unit (50), wherein the first computer unit (50) comprises:
a first user interface (51) configured to display information to the user, for example, configured to display instructions on a palpation examination tutorial and receive input from the user, wherein the first computer unit (50) is selected from the group comprised of: portable computer, fixed computer, tablet, smartphone, smartwatch;
wherein the first computer unit (50) is configured to communicate with the power and control unit (20) of the palpation glove (10) and a second computer unit (90), by wired or wireless communication means;
second computer unit (90), wherein the second computer unit (90) is a device selected from the group comprised of: server, local network computer, remote cloud computer, portable computer, fixed computer, tablet, smartphone or any other computing device accessible through a communications network such as, for example, the Internet, wherein the second computer unit (90) is configured to communicate with the first computer unit (50) by wired or wireless communication means.

11. The system (300) according to claim 10, characterized in that the second computer unit (90) is integrated in the power and control unit (20) of the palpation glove (10) or is integrated in the first computer unit (50).

12. The system (300) according to claim 10, characterized in that the pressure sensors (15) of the palpation glove (10) are arranged so as to keep between them a distance d, wherein d pertains to the interval 0 cm to 2.5 cm and preferably d is 0.1 cm.

13. The system (300) according to claim 10, characterized in that the palpation glove (10) comprise, on its main body (17A) two side openings for the thumb, so as to enable the user to utilize the palpation glove (10) with the left hand or with the right hand, and wherein the sensors (15) have an arrangement on the board (14) which is symmetrical relative to a central vertical axis of the flexible board (14) of the palpation glove (10).

14. The system (300) according to claim 10, characterized in that the palpation glove (10) further comprises a movement sensor (23), wherein the movement sensor (23) is placed on the board (14) or is placed on the power and control unit (20) and wherein the movement sensor (23) is selected from at least one of the group comprised of an accelerometer or a gyroscope.

15. The system (300) according to claim 10, characterized in that the palpation glove (10) further comprises a mark configured to assist the user to position the glove correctly on the reference point (7) of the patient's body part at the start of each palpation examination.

16. The system (300) according to claim 10, characterized in that at least one light source (12) is configured to emit, permanently or intermittently, one or more indicative colors, wherein the colors of the light source indicate at least one of the following statuses of the palpation glove (10):
turned off status;
turned on status awaiting new command;
status ready to start palpation examination;
self-calibration;
palpation examination concluded;
power supply (21) with low charge;
power supply (21) charging;
power supply (21) charged, palpation glove (10) ready to operate.

17. A computer program product comprising a set of logical instructions which, when the program is run by a computer, the computer performs the steps j) to l) of the method as defined in claim 1.

18. A computer-readable storage means comprising a set of logical instructions which, when run by a computer, the computer performs the steps j) to l) of the method as defined in claim 1.
